(19) United States
(12) Patent Application Publication
(10) Pub. No.: US 2026/0355573 A1
(43) Pub. Date: Nov. 12, 2026

(54) MOTION PREDICTION APPARATUS, MOTION PREDICTION METHOD, AND PROGRAM

(71) Applicant: NEC Corporation, Tokyo (JP)

(72) Inventor: Kosuke NISHIHARA, Tokyo (JP)

(73) Assignee: NEC Corporation, Tokyo (JP)

(21) Appl. No.: 19/514,953

(22) PCT Filed: Sep. 11, 2023
(86) PCT No.: PCT/JP2023/033047

§ 371 Date: Feb. 27, 2026

(51) Int. Cl. A63B 24/00; A63B 71/06; G06T 7/246

(52) U.S. Cl. CPC A63B 24/0003; G06T 7/246; A63B 2071/065; A63B 2220/05; G06T 2207/10016; G06T 2207/20081; G06T 2207/30196

(57) ABSTRACT

A motion prediction apparatus 100 in the present disclosure includes: an extracting unit 121 that extracts motion feature data representing a feature of a motion from motion data of a person; and a predicting unit 122 that predicts predicted motion feature data representing a change in the motion feature data based on exercise data representing content of exercise performed by a person and on the motion feature data. Then, the extracting unit 121 and the predicting unit 122 can be constructed with artificial intelligence by machine learning and can support decision-making of users using a prediction result.

### TECHNICAL FIELD

The present disclosure relates to a motion prediction apparatus, a motion prediction method, and a program.

### BACKGROUND ART

Exercise and training may be performed with the expectation of motion improvements such as long-term care prevention and frailty prevention. At this time, when the result of motion improvements through exercise and training can be predicted in advance, it will motivate a person to perform exercise and the like. For example, in Patent Literature 1, the amount of exercise is predicted from walking images for the purpose of health management.

### CITATION LIST

### Patent Literature

Patent Literature 1: Japanese Unexamined Patent Application Publication JP-A 2018-099267

### SUMMARY OF INVENTION

### Technical Problem

However, the technique described in Patent Literature 1 described above merely predicts the amount of exercise through walking and cannot predict a change in motion itself through exercise. Therefore, there arises a problem that a change in motion through exercise cannot be predicted appropriately.

Accordingly, an object of the present disclosure is to solve the abovementioned problem that a change in motion through exercise cannot be predicted appropriately.

### Solution to Problem

A motion prediction apparatus as an aspect of the present disclosure includes: an extracting unit configured to extract motion feature data representing a feature of a motion from motion data of a person; and a predicting unit configured to predict predicted motion feature data representing a change in the motion feature data based on exercise data representing content of exercise performed by a person and on the motion feature data.

Further, a motion prediction method as an aspect of the present disclosure includes: extracting motion feature data representing a feature of a motion from motion data of a person; and predicting predicted motion feature data representing a change in the motion feature data based on exercise data representing content of exercise performed by a person and on the motion feature data.

Further, a program as an aspect of the present disclosure includes instructions for causing a computer to execute processes to: extract motion feature data representing a feature of a motion from motion data of a person; and predict predicted motion feature data representing a change in the motion feature data based on exercise data representing content of exercise performed by a person and on the motion feature data.

### ADVANTAGEOUS EFFECTS OF INVENTION

With the configurations as described above, the present disclosure allows appropriate prediction of a change in motion through exercise.

### BRIEF DESCRIPTION OF DRAWINGS

FIG. 1 is a block diagram showing a configuration of a motion prediction apparatus according to the present disclosure.

FIG. 2 is a diagram showing a state of processing by the motion prediction apparatus according to the present disclosure.

FIG. 3 is a flowchart showing processing operation of the motion prediction apparatus according to the present disclosure.

FIG. 4 is a diagram showing a state of processing by the motion prediction apparatus according to the present disclosure.

FIG. 5 is a diagram showing a state of processing by the motion prediction apparatus according to the present disclosure.

FIG. 6 is a flowchart showing processing operation of the motion prediction apparatus according to the present disclosure.

FIG. 7 is a diagram showing a configuration and a state of processing of a motion prediction apparatus according to the present disclosure.

FIG. 8 is a block diagram showing a hardware configuration of the motion prediction apparatus according to the present disclosure.

FIG. 9 is a block diagram showing a configuration of the motion prediction apparatus according to the present disclosure.

### EXAMPLE EMBODIMENT

### First Example Embodiment

A first example embodiment of the present disclosure will be described with reference to the drawings. The drawings may be related to any of example embodiments. A motion prediction apparatus 10 in this example embodiment predicts the motion of a person that can change from the motion data of the person and motion data that the person can perform. For example, the motion prediction apparatus 10 is used in the field of healthcare and predicts a motion that can be improved through exercise and training that can be performed with the expectation of improvement in motion for long-term care prevention and frailty prevention of a person. At this time, the motion of a person include, for example, a walk and a sit-to-stand motion, and in the following description, five sit-to-stand motions will be taken as an example. However, a motion targeted by the motion prediction apparatus 10 may be any kind of motion, and the motion prediction apparatus 10 is not limited to being used in the field of healthcare and may be used for prediction of a change in any motion in any field.

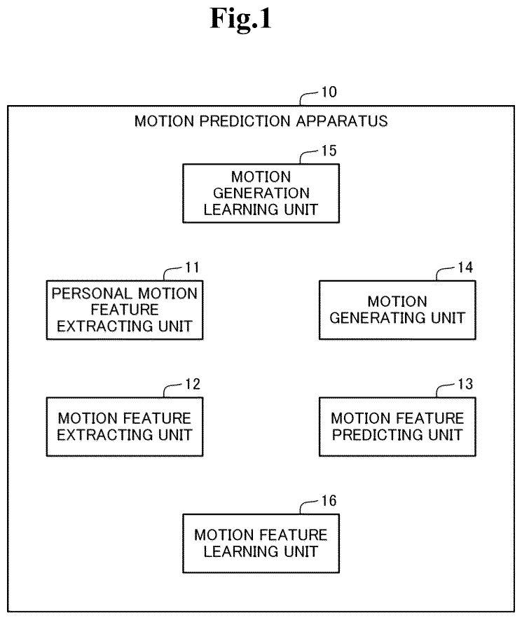

The motion prediction apparatus 10 is configured with one or a plurality of information processing apparatuses each including an arithmetic logic unit and a memory unit. Then, as shown in FIG. 1, the motion prediction apparatus 10 includes a personal motion feature extracting unit 11, a motion feature extracting unit 12, a motion feature predicting unit 13, a motion generating unit 14, a motion generation learning unit 15, and a motion feature learning unit 16. The respective functions of the motion feature extracting unit 11, the motion feature extracting unit 12, the motion feature predicting unit 13, the motion generating unit 14, the motion generation learning unit 15, and the motion feature learning unit 16 can be implemented by execution of a program for implementing the respective functions stored in the memory unit by the arithmetic logic unit. Below, the respective components will be described in detail along with the operation of the motion prediction apparatus 10. In the following, an inference operation to predict a motion by the motion prediction apparatus 10 will be described first, and then, a learning operation for constructing the motion prediction apparatus 10 by machine learning in such a manner as to enable the inference operation will be described.

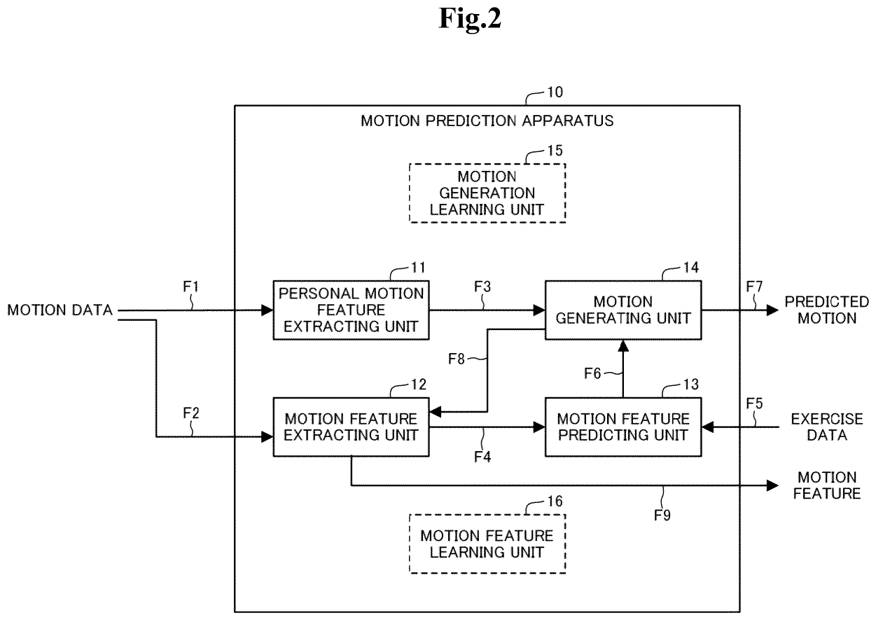

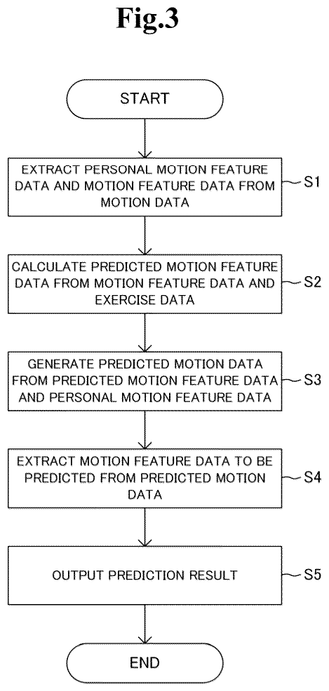

FIG. 2 is a block diagram showing a configuration of the motion prediction apparatus 10, showing the flow of data during the inference operation. Further, FIG. 3 is a flowchart showing the inference operation of the motion prediction apparatus 10.

The personal motion feature extracting unit 11 and the motion feature extracting unit 12 (extracting unit) first acquire the motion data of a person to be the target for motion prediction (F1 and F2 in FIG. 2). At this time, the person predicts a motion that can be improved through exercise and training that can be performed with the expectation of improvement in motion for long-term care prevention and frailty prevention, for example. At this time, the person to be the target is, for example, an elderly person, and the motion prediction is performed for long-term care prevention and frailty prevention. Then, the motion data of the person is, for example, data extracted from video data obtained by capturing five sit-to-stand motions by the person. A specific example of the motion data includes time-series data of the coordinate values (e.g. (x, y, z)) and the rotation angle (e.g. Euler angle) of each joint of the person extracted from the video data of 64 consecutive frames. However, the motion data input into the motion prediction apparatus 10 may be video data itself, and the motion data such as the coordinate values of each joint described above may be extracted from the video data by the personal motion feature extracting unit 11 and the motion feature extracting unit 12.

Then, the personal motion feature extracting unit 11 and the motion feature extracting unit 12 extract vector data obtained by digitizing motion features from the motion data such as the coordinate values input thereto, respectively (step S1 in FIG. 3). To be specific, the personal motion feature extracting unit 11 and the motion feature extracting unit 12 each extract a motion time, a difference in time between the first and last occurrences of periodic motion, joint angles of the back and knee in the standing position, exercise ranges of the back and knee during motion, left and right balance and unsteadiness, and the operation order of each joint, in the form of vector data as the motion features from the motion data. In this example embodiment, a case where the degree of the maximum curvature of the back during the sit-to-stand motion (hereinafter referred to as “maximum curvature”) is extracted as the motion feature will be described as an example. At this time, the degree of the maximum curvature as the motion feature to be extracted may be a continuous value or a discrete value, but in this example embodiment, it is a discrete value divided into five levels. That is to say, the personal motion feature extracting unit 11 and the motion feature extracting unit 12 extract vector data that the maximum curvature of the back during the sit-to-stand motion corresponds to any one of the five levels. The vector data is, for example, vector data having elements such as 256 elements or 512 elements obtained by dimensionally compressing the motion data.

Here, the personal motion feature extracting unit 11 extracts personal motion feature vector data (personal motion feature data) representing a motion feature of each individual person from the motion data. That is to say, the personal motion feature extracting unit 11 extracts personal motion feature vector data in such a manner that it becomes approximate vector data even if the motion is any kind of motion when the motion is that of the same person regardless of the feature of the motion itself. That is to say, in this example embodiment, even if the maximum curvature of the input motion data is equivalent to any value when the motion is that of the same person, approximate vector data is output. Then, the personal motion feature extracting unit 11 outputs the extracted personal motion feature vector data to the motion generating unit 14 (F3 in FIG. 2).

The personal motion feature extracting unit 11 is configured with a machine learning model that is generated in advance by machine learning in such a manner as to extract personal motion feature vector data from motion data as described above. For example, the machine learning model configuring the personal motion feature extracting unit 11 is generated through machine learning in such a manner as to, with training motion data and training labels that are values of 1 to 5 representing the degree of the maximum curvature corresponding to the motion that are acquired from a person in advance as training data, compare personal motion feature vectors extracted from the motion data and the training motion data, respectively, using a loss function and, when they are of the same person, optimize the weight of a neural network configuring the machine learning model in such a manner that the vector data are approximated.

However, the personal motion feature extracting unit 11 may be a machine learning model generated by machine learning by any method, and it is not necessarily limited to being configured with a machine learning model and may be configured in any manner, such as being configured with an arithmetic formula set in advance.

Further, the motion feature extracting unit 12 extracts motion feature vector data (motion feature data) representing the motion feature of a motion itself from motion data. That is to say, the personal motion feature extracting unit 11 extracts the motion feature vector data in such a manner that it becomes approximate vector data even if the motion is any kind of motion when the motion is that of the same person regardless of the motion feature of an individual person. That is to say, in this example embodiment, even if a person of input motion data is different, when the value of the maximum curvature is equivalent to the same value, approximate vector data is output. Then, the motion feature extracting unit 12 outputs the extracted motion feature vector data to the motion feature predicting unit 13 (F4 of FIG. 2).

The motion feature extracting unit 12 is configured with a machine learning model generated in advance by machine learning in such a manner as to extract motion feature vector data from motion data as described above. For example, the machine learning model configuring the motion feature extracting unit 12 is generated by machine learning in such a manner as to, with the abovementioned training motion data and training labels that are acquired from a plurality of persons in advance as training data, compare motion feature vectors extracted from the motion data and the training motion data, respectively, using a loss function and, when they are of the same training label, optimize the weight of a neural network configuring the machine learning model in such a manner that the vector data are approximated. However, the motion feature extracting unit 12 may be a machine learning model generated by machine learning by any method, and it is not necessarily limited to being configured with a machine learning model and may be configured in any manner such as being configured with an arithmetic formula set in advance.

Subsequently, the motion feature predicting unit 13 (predicting unit) acquires exercise data of a person to be the target for motion prediction (F5 of FIG. 2). At this time, the exercise data of a person is data representing the content of exercise that can be performed by the person, such as data representing the content of strength training, aerobic exercise, and balance training. As a specific example of the exercise data may be vector data obtained by digitizing leg press (load, repetitions, frequency), treadmill (speed, time, frequency), single-leg standing (time, frequency) and the like, or may be data in binary values of (1, 0) representing whether exercise corresponding to the training name was performed.

Then, the motion feature predicting unit 13 predicts predicted motion feature data representing a change in motion of a person to be the target for motion prediction based on the motion feature vector data output from the motion feature extracting unit 12 and the motion data (step S2 of FIG. 3). To be specific, the motion feature predicting unit 13 calculates predicted motion feature vector data that is vector data representing a motion after the change, from vector data obtained by combining the motion feature vector data and exercise vector data that is exercise data. Then, the motion feature predicting unit 13 outputs the predicted motion feature vector data to the motion generating unit 14 (F6 of FIG. 2).

Here, the motion feature predicting unit 13 is configured with a machine learning model generated in advance by machine learning in such a manner as to predict the predicted motion feature data from the motion feature vector data and the exercise data as described above. For example, the machine learning model configuring the motion feature predicting unit 13 is generated through machine learning in such a manner as to, with the training motion data and the training labels as described above as training data, optimize the weight of a neural network in such a manner that predicted motion feature data having been predicted matches the training labels, or in such a manner that the predicted motion feature data predicted from the motion data and the training motion data, respectively, match. However, the motion feature predicting unit 13 may be a machine learning model generated by machine learning by any method, and it is not necessarily limited to being configured with a machine learning model and may be configured in any manner, such as being configured with an arithmetic formula set in advance.

Subsequently, the motion generating unit 14 (generating unit) generates predicted motion data that is the motion data to be predicted of a person based on the predicted motion feature data and the personal motion feature data (step S3 of FIG. 3). To be specific, the motion generating unit 14 calculates predicted motion data representing a predicted motion after change from vector data obtained by combining the predicted motion feature vector data and the personal motion feature vector data. As a specific example, the predicted motion data is time-series vector data of the coordinate values (e.g. (x, y, z)) and the rotation angle (e.g. Euler angle) of each joint of a person in the same manner as the motion data described above. Then, the motion generating unit 14 outputs the generated predicted motion data (F7 in FIG. 2). At this time, the motion generating unit 14 generates video data in which the person is performing the predicted motion, that is, the sit-to-stand motion from the time-series predicted motion data as described above, and outputs the video data (step S5 in FIG. 3).

Here, the motion generating unit 14 is configured with a machine learning model generated in advance by machine learning in such a manner as to generate the predicted motion data from the predicted motion feature data and the personal motion feature data as described above. For example, the machine learning model configuring the motion generating unit 14 is generated through machine learning in such a manner as to, with the training motion data and the training labels as described above as training data, optimize the weight of a neural network in such a manner that predicted motion data to be generated matches the training motion data. However, the motion generating unit 14 may be a machine learning model generated by machine learning by any method, and it is not necessarily limited to being configured with a machine learning model an may be configured in any manner, such as being configured with an arithmetic formula set in advance.

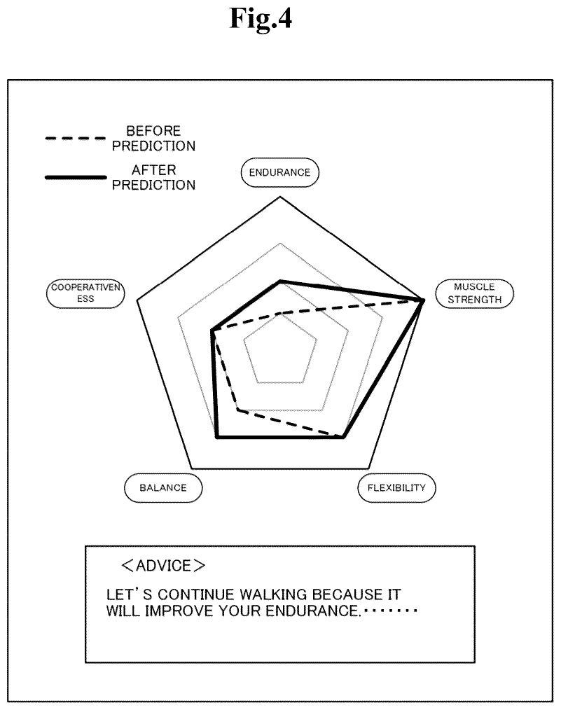

Here, the abovementioned motion feature extracting unit 12 (extracting unit) receives as an input the predicted motion data generated by the motion generating unit 14 (F8 in FIG. 2), and extracts motion feature data after the prediction (second predicted motion feature data) representing the motion feature of the predicted motion data from the predicted motion data (step S4 in FIG. 3). At this time, the motion feature extracting unit 12 extracts motion feature vector data representing the motion feature after the prediction from vector data that is the predicted motion data in the same manner as described above. Then, the motion feature extracting unit 12 outputs the calculated motion feature data after the prediction. At this time, the motion feature extracting unit 12 outputs comparison data obtained by comparing the motion feature data extracted from the motion data before the prediction with the motion feature data after the prediction (step S5 of FIG. 3). For example, the motion feature extracting unit 12 extracts a feature regarding a motion of a person that can be further extracted from each of the motion feature data before and after the prediction, and outputs the features in such a manner as to be comparable before and after the prediction. Examples of a feature that can be extracted from the motion feature data include endurance, strength, flexibility, and balance. In this case, for example, as shown in FIG. 4, the feature before the prediction (dotted line) and the feature after the prediction (solid line) are compared and displayed on the same graph. The motion feature extracting unit 12 can also generate and output advice data corresponding to the result of comparison of the features before and after the prediction as shown in FIG. 4. For example, the motion feature extracting unit 12 may hold advice data corresponding to each change in the feature in advance, and as a result of actually predicting the motion of a person, select and output advice data corresponding to a change in the feature.

As described above, the motion prediction apparatus 10 according to this example embodiment can appropriately predict a subsequent motion from the motion data of a person and exercise data. Then, for example, by using the motion prediction apparatus 10 in the medical and healthcare fields, it is possible to predict improvement in motion through exercise and training in order to prevent long-term care and frailty of a target person. In this case, a doctor and a medical professional who provide instructions on long-term care prevention and frailty prevention to a target person refer to the prediction result from the motion prediction apparatus 10, thereby assisting decision-making regarding instructions on the content of exercise and training.

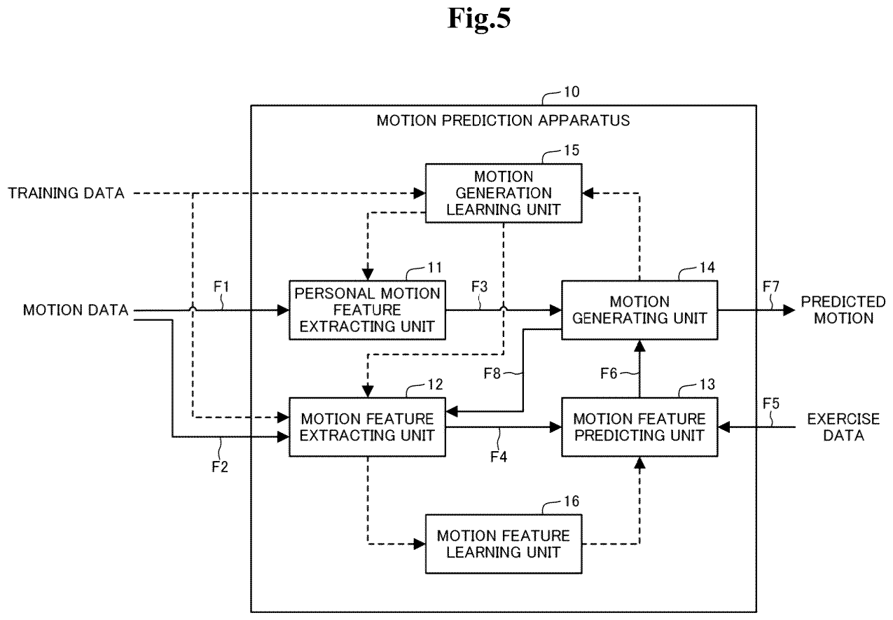

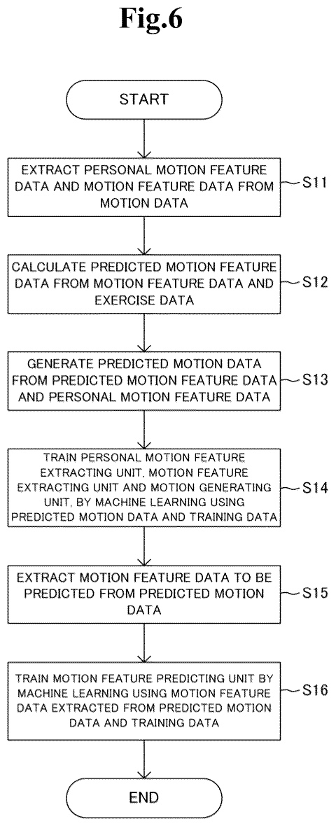

Next, a configuration for constructing the abovementioned motion prediction apparatus 10 by machine learning and the learning operation will be described. FIG. 5 is a block diagram showing a configuration of the motion prediction apparatus 10, showing the flow of data during the learning operation. Further, FIG. 6 is a flowchart showing the learning operation of the motion prediction apparatus 10.

First, when the motion prediction apparatus 10 performs machine learning, training data is prepared that corresponds to the motion data of a person described above and includes training motion data representing a subsequent motion after change of the person and a training label corresponding to the motion.

In the same manner as described before, in the motion prediction apparatus 10, the personal motion feature extracting unit 11 and the motion feature extracting unit 12 (extracting unit) extract personal motion feature data and motion feature data, with the motion data of a person as inputs (F1 and F2 in FIG. 5) (step S11 in FIG. 6). Subsequently, in the same manner as described before, in the motion prediction apparatus 10, the motion feature predicting unit 13 (predicting unit) calculates predicted motion feature data, with the motion feature data and exercise data as inputs (F4 and F5 in FIG. 5) (step S12 in FIG. 6). Furthermore, in the same manner as described before, in the motion prediction apparatus 10, the motion generating unit 14 (generating unit) generates predicted motion data, with the personal motion feature data and the predicted motion feature data as inputs (F3 and F6 in FIG. 5) (step S13 in FIG. 6).

Then, the motion generation learning unit 15 (learning unit) performs machine learning so as to optimize the weights of neural networks configuring the personal motion feature extracting unit 11, the motion feature extracting unit 12, and the motion generating unit 14, respectively, using the generated predicted motion data and training data as indicated by a dotted line in FIG. 5. At this time, the motion generation learning unit 15 may compare the predicted motion data generated from the motion data with training motion data of the training data using a loss function and perform machine learning so as to optimize the weight of the neural network configuring the motion generating unit 14. Moreover, the motion generation learning unit 15 may compare the personal motion feature data and the motion feature data extracted from the motion data with the personal motion feature data and the motion feature data extracted from the training motion data of the training data using a loss function and perform machine learning so as to optimize the weights of the neural networks configuring the personal motion feature extracting unit 11 and the motion feature extracting unit 12. However, the motion generation learning unit 15 may train by machine learning the neural networks configuring the personal motion feature extracting unit 11, the motion feature extracting unit 12, and the motion generating unit 14 by any method. Moreover, the motion generation learning unit 15 is not limited to performing learning on all of the personal motion feature extracting unit 11, the motion feature extracting unit 12, and the motion generating unit 14, and may perform learning on only one of them.

Further, the motion feature learning unit 16 (learning unit) performs machine learning so as to optimize the weight of a neural network configuring the motion feature predicting unit 13, using the generated predicted motion data and the training data as indicated by a dotted line in FIG. 5. At this time, the motion feature learning unit 16 may compare the predicted motion feature data to be predicted based on the motion feature data extracted from the generated predicted motion data with the predicted motion feature data to be predicted based on the motion feature data extracted from the training motion data using a loss function, and perform machine learning so as to optimize the weight of the neural network configuring the motion feature predicting unit 13. Alternatively, the motion feature learning unit 16 may train by machine learning the motion feature predicting unit 13 by comparing the predicted motion feature data predicted from the motion data described above with training labels of the training data using a loss function. However, the motion feature learning unit 16 may train by machine learning the neural network configuring the motion feature predicting unit 13 by any method.

Here, the learning processes by the motion generation learning unit 15 and the motion feature learning unit 16 described above may be performed simultaneously. This allows for machine learning to be performed using more information than when each machine learning model is trained by machine learning independently, which is expected to result in higher prediction accuracy for each model.

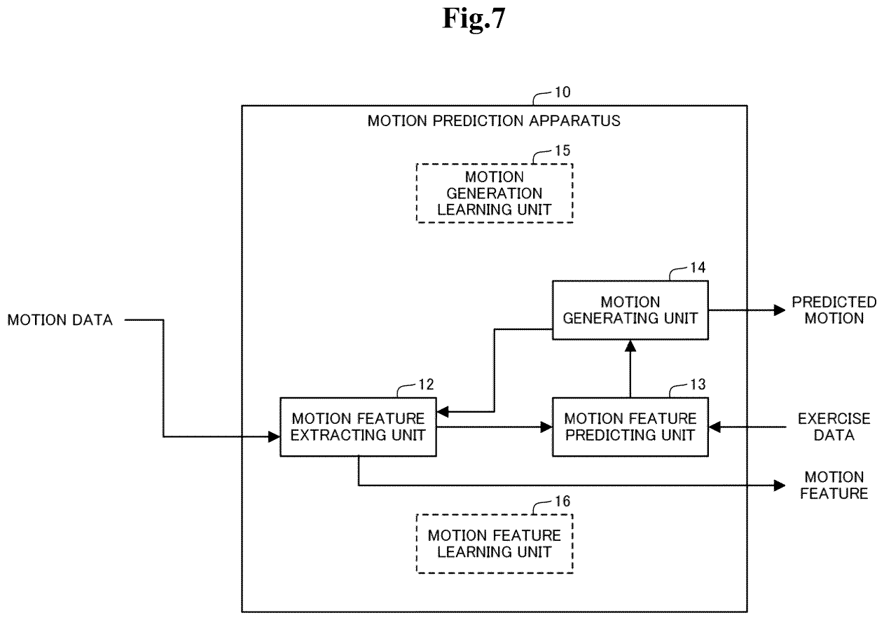

As shown in FIG. 7, the motion prediction apparatus 10 in this example embodiment does not necessarily have to include the personal motion feature extracting unit 11 described above. According to the motion prediction apparatus 10 shown in FIG. 7, at the time of inference operation, first, the motion feature extracting unit 12 (extracting unit) extracts motion feature data using the motion data of a person as an input, and the motion feature predicting unit 13 (predicting unit) calculates predicted motion feature data using the motion feature data and exercise data as inputs. Then, the motion generating unit 14 (generating unit) generates predicted motion data using the personal motion feature data as an input. Furthermore, the motion feature extracting unit 12 extracts motion feature data after prediction from the generated predicted motion data.

Further, in this case, at the time of learning operation, the motion generation learning unit 15 and the motion feature learning unit 16 train by machine learning the motion feature extracting unit 12, the motion generating unit 14 and the motion feature predicting unit 13 using predicted motion data and training data in the same manner as described above.

### Second Example Embodiment

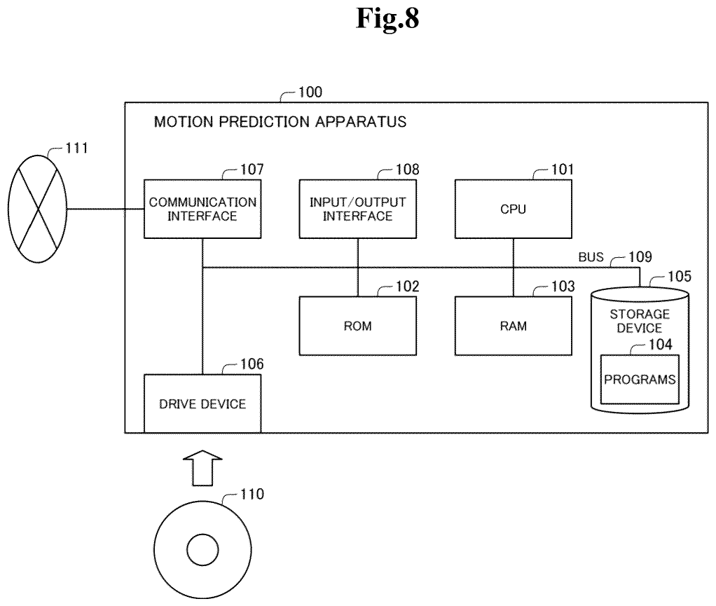

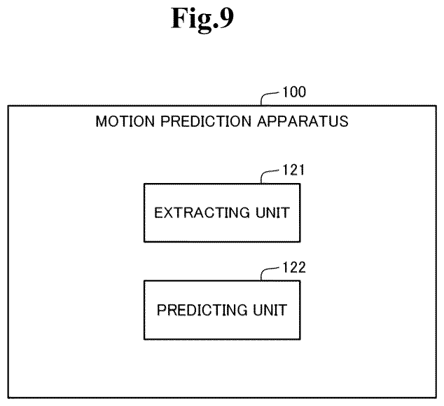

Next, a second example embodiment of the present disclosure will be described with reference to the drawings. In this example embodiment, the overview of the configuration of the motion prediction apparatus described in the above example embodiment is shown. FIGS. 8 to 9 are diagrams for describing the configuration, and the drawings may be associated with any of the example embodiments.

First, a hardware configuration of a motion prediction apparatus 100 will be described with reference to FIG. 8. The motion prediction apparatus 100 is configured with a general-purpose information processing apparatus and, as an example, has the following hardware configuration including:
a CPU (Central Processing Unit) 101 (arithmetic logic unit);
a ROM (Read Only Memory) 102 (memory unit);
a RAM (Random Access Memory) 103 (memory unit);
programs 104 loaded into the RAM 103;
a storage device 105 storing the programs 104;
a drive device 106 that performs reading from and writing into a storage medium 110 external to the information processing apparatus;
a communication interface 107 connected to a communication network 111 external to the information processing apparatus;
an input/output interface 108 that performs input/output of data; and
a bus 109 connecting the components.

FIG. 8 shows an example of the hardware configuration of an information processing apparatus serving as the motion prediction apparatus 100, and the hardware configuration of the information processing apparatus is not limited to the abovementioned case. For example, the information processing apparatus may be configured with part of the abovementioned configuration, such as not having the drive device 106. Moreover, the information processing apparatus may use a GPU (Graphic Processing Unit), a DSP (Digital Signal Processor), an MPU (Micro Processing Unit), an FPU (Floating point number Processing Unit), a PPU (Physics Processing Unit), a TPU (Tensor Processing Unit), a quantum processor, a microcontroller, or a combination thereof, instead of the abovementioned CPU.

Then, the motion prediction apparatus 100 can construct and include an extracting unit 121 and a predicting unit 122 shown in FIG. 9 by acquisition and execution of the programs 104 by the CPU 101. The programs 104 are, for example, stored in advance in the storage device 105 or the ROM 102, and are loaded into the RAM 103 and executed by the CPU 101 as necessary. Moreover, the programs 104 may be provided to the CPU 101 via the communication network 111, or the programs may be stored in advance in the storage medium 110 and read out by the drive device 106 and provided to the CPU 101. However, the extracting unit 121 and predicting unit 122 described above may be constructed using dedicated electronic circuits for implementing such means.

The extracting unit 121 extracts motion feature data representing the feature of a motion from the motion data of a person. Further, the predicting unit 122 predicts predicted motion feature data representing a change in the motion feature data based on exercise data representing the content of exercise performed by a person and the motion feature data.

Configured as described above, the present disclosure can appropriately predict a change in motion itself due to exercise.

At least one or more functions of the functions of the extracting unit 121 and the predicting unit 122 described above may be executed by an information processing apparatus installed and connected anywhere on the network, that is, may be executed by so-called cloud computing.

Further, the abovementioned programs can be stored using various types of non-transitory computer-readable mediums and provided to a computer. The non-transitory computer-readable medium includes various types of tangible storage mediums.

Examples of non-transitory computer-readable medium include magnetic recording medium (e.g., flexible disk, magnetic tape, hard disk drive), magneto-optical recording medium (e.g., magneto-optical disk), read only memory (CD-ROM), CD-R, CD-R/W, semiconductor memory (e.g., mask ROM, programmable ROM, Erasable PROM, flash ROM, random access memory (RAM)). In addition, a program may be provided to a computer by various types of temporary computer-readable medium. Examples of temporary computer-readable medium include electrical signals, optical signals, and electromagnetic waves. The temporary computer-readable medium may provide a program to the computer via a wired communication channel, such as an electric wire and an optical fiber, or a wireless communication channel.

Although the present disclosure has been described above with reference to the above-described example embodiments, the present disclosure is not limited to the embodiments described above. The configuration and details of the present disclosure can be changed in a variety of ways that those skilled in the art can understand within the scope of the present disclosure. Then, each of the example embodiments described above can be combined with the other example embodiment as necessary.

### Supplementary Notes

The whole or part of the example embodiments disclosed above can be described as the following supplementary notes. Hereinafter, the overview of the configurations of a motion prediction apparatus, a motion prediction method, and a program in the present disclosure will be described. However, the present disclosure is not limited to the following configurations.

### (Supplementary Note 1)

A motion prediction apparatus comprising:
an extracting unit configured to extract motion feature data representing a feature of a motion from motion data of a person; and
a predicting unit configured to predict predicted motion feature data representing a change in the motion feature data based on exercise data representing content of exercise performed by a person and on the motion feature data.

### (Supplementary Note 2)

The motion prediction apparatus according to supplementary note 1, wherein the extracting unit is configured to extract from the motion data a plurality of features set in accordance with motions as the motion feature data.

### (Supplementary Note 3)

The motion prediction apparatus according to supplementary note 1, comprising a generating unit configured to generate predicted motion data that is motion data to be predicted of a person based on the predicted motion feature data.

### (Supplementary Note 4)

The motion prediction apparatus according to supplementary note 3, wherein the extracting unit is configured to extract second predicted motion feature data representing a feature of a motion from the predicted motion data.

### (Supplementary Note 5)

The motion prediction apparatus according to supplementary note 1, wherein the extracting unit is configured to extract personal motion feature data representing a motion feature of a person who has performed a motion from the motion data, the motion prediction apparatus comprising a generating unit configured to generate predicted motion data that is motion data to be predicted of a person based on the predicted motion feature data and the personal motion feature data.

### (Supplementary Note 6)

The motion prediction apparatus according to supplementary note 5, wherein the extracting unit is configured to extract from the motion data a plurality of features set in accordance with motions as the personal motion feature data.

### (Supplementary Note 7)

The motion prediction apparatus according to supplementary note 5, wherein the extracting unit is configured to extract second predicted motion feature data representing a feature of a motion from the predicted motion data.

### (Supplementary Note 8)

The motion prediction apparatus according to supplementary note 5, comprising a learning unit configured to train by machine learning at least one of the extracting unit, the predicting unit, and the generating unit, using training data including training motion data of a person and a training label corresponding to the training motion data of the person and the predicted motion data.

### (Supplementary Note 9)

A motion prediction method comprising:
extracting motion feature data representing a feature of a motion from motion data of a person; and
predicting predicted motion feature data representing a change in the motion feature data based on exercise data representing content of exercise performed by a person and on the motion feature data.

### (Supplementary Note 10)

The motion prediction method according to supplementary note 9, comprising generating predicted motion data that is motion data to be predicted of a person based on the predicted motion feature data.

### (Supplementary Note 11)

The motion prediction method according to supplementary note 10, comprising extracting second predicted motion feature data representing a feature of a motion from the predicted motion data.

(supplementary Note 12)

The motion prediction method according to supplementary note 9, comprising:
extracting personal motion feature data representing a motion feature of a person who has performed a motion from the motion data; and
generating predicted motion data that is motion data to be predicted of a person based on the predicted motion feature data and the personal motion feature data.

### (Supplementary Note 13)

The motion prediction method according to supplementary note 12, comprising extracting second predicted motion feature data representing a feature of a motion from the predicted motion data.

(supplementary Note 14)

The motion prediction method according to supplementary note 12, comprising training by machine learning at least one of a model extracting the motion feature data, a model extracting the personal motion feature data, and a model predicting the predicted motion feature data, using training data including training motion data of a person and a training label corresponding to the training motion data of the person and the predicted motion data.

### (Supplementary Note 15)

A non-transitory computer-readable storage medium storing a program, the program comprising instructions for causing a computer to execute processes to:
extract motion feature data representing a feature of a motion from motion data of a person; and
predict predicted motion feature data representing a change in the motion feature data based on exercise data representing content of exercise performed by a person and on the motion feature data.

### REFERENCE SIGNS LIST

10 motion prediction apparatus
11 personal motion feature extracting unit
12 motion feature extracting unit
13 motion feature predicting unit
14 motion generating unit
15 motion generation learning unit
16 motion feature learning unit
100 motion prediction apparatus
101 CPU
102 ROM
103 RAM
104 programs
105 storage device
106 drive device
107 communication interface
108 input/output interface
109 bus
110 storage medium
111 communication network
121 extracting unit
122 predicting unit

What is claimed is:

1. A motion prediction apparatus comprising:
a sensor configured to acquire motion data representing a physical motion of a person;
at least one memory storing processing instructions; and
at least one processor configured to execute the processing instructions, wherein the at least one processor is configured to execute the processing instructions to:
extract motion feature data representing a feature of a motion from the motion data acquired by the sensor;
predict predicted motion feature data representing a change in the motion feature data based on exercise data representing content of exercise performed by a the person and on the motion feature data; and
generate, based on the predicted motion feature data, predicted video data for displaying a predicted physical motion of the person.

2. The motion prediction apparatus according to claim 1, wherein the at least one processor is configured to execute the processing instructions to
extract from the motion data a plurality of features set in accordance with motions as the motion feature data.

3. The motion prediction apparatus according to claim 1, wherein the at least one processor is configured to execute the processing instructions to
generate predicted motion data that is motion data to be predicted of a person based on the predicted motion feature data.

4. The motion prediction apparatus according to claim 3, wherein the at least one processor is configured to execute the processing instructions to
extract the second predicted motion feature data representing a feature of a motion from the predicted motion data.

5. The motion prediction apparatus according to claim 1, wherein the at least one processor is configured to execute the processing instructions to:
extract personal motion feature data representing a motion feature of a person who has performed a motion from the motion data; and
generate predicted motion data that is motion data to be predicted of a person based on the predicted motion feature data and the personal motion feature data.

6. The motion prediction apparatus according to claim 5, wherein the at least one processor is configured to execute the processing instructions to
extract from the motion data a plurality of features set in accordance with motions as the personal motion feature data.

7. The motion prediction apparatus according to claim 5, wherein the at least one processor is configured to execute the processing instructions to
extract second predicted motion feature data representing a feature of a motion from the predicted motion data.

8. The motion prediction apparatus according to claim 5, wherein the at least one processor is configured to execute the processing instructions to
train by machine learning at least one of a model extracting the motion feature data, a model extracting the personal motion feature data, and a model predicting the predicted motion feature data, using training data including training motion data of a person and a training label corresponding to the training motion data of the person and the predicted motion data.

9. A motion prediction method comprising:
acquiring, via a sensor, motion data representing a physical motion of a person;
extracting motion feature data representing a feature of a motion from the motion data acquired by the sensor;
predicting predicted motion feature data representing a change in the motion feature data based on exercise data representing content of exercise performed by a the person and on the motion feature data; and
generating, based on the predicted motion feature data, predicted video data for displaying a predicted physical motion of the person.

10. The motion prediction method according to claim 9, comprising
generating predicted motion data that is motion data to be predicted of a person based on the predicted motion feature data.

11. The motion prediction method according to claim 10, comprising
extracting second predicted motion feature data representing a feature of a motion from the predicted motion data.

12. The motion prediction method according to claim 9, comprising:
extracting personal motion feature data representing a motion feature of a person who has performed a motion from the motion data; and
generating predicted motion data that is motion data to be predicted of a person based on the predicted motion feature data and the personal motion feature data.

13. The motion prediction method according to claim 12, comprising
extracting second predicted motion feature data representing a feature of a motion from the predicted motion data.

14. The motion prediction method according to claim 12, comprising
training by machine learning at least one of a model extracting the motion feature data, a model extracting the personal motion feature data, and a model predicting the predicted motion feature data, using training data including training motion data of a person and a training label corresponding to the training motion data of the person and the predicted motion data.

15. A non-transitory computer-readable storage medium storing a program, the program comprising instructions for causing a computer to execute processes to:
receiving motion data from a sensor, the motion data representing a physical motion of a person;
extract motion feature data representing a feature of a motion from the motion data; and
predict predicted motion feature data representing a change in the motion feature data based on exercise data representing content of exercise performed by the person and on the motion feature data-; and
generate, based on the predicted motion feature data, predicted video data for displaying a predicted physical motion of the person.
